(19) United States
(12) Patent Application Publication
(10) Pub. No.: US 2014/0020645 A1
(43) Pub. Date: Jan. 23, 2014

(54) LUBRICANT COMPOSITIONS FOR DIRECT INJECTION ENGINES

(71) Applicants: Gregory H. GUINTHER, Richmond, VA (US); John T. LOPER, Richmond, VA (US)

(72) Inventors: Gregory H. GUINTHER, Richmond, VA (US); John T. LOPER, Richmond, VA (US)

(73) Assignee: AFTON CHEMICAL CORPORATION, Richmond, VA (US)

(21) Appl. No.: 13/551,836

(22) Filed: Jul. 18, 2012

(51) Int. Cl. C10M 129/10; C07C 39/07; F02B 77/00; C07C 39/06

(52) U.S. Cl. 123/1 A; 508/584; 568/784

(57) ABSTRACT

A lubricant additive, an crankcase lubricant composition and a method for reducing intake valve deposits in a spark ignition direct injection (SIDI) engine. The lubricant additive includes an aromatic compound having a boiling point under standard atmospheric conditions of from about 190° to about 270° C. The aromatic compound is effective to reduce intake valve deposits in a SIDI engine when used in an amount ranging from about 0.1 to about 5.0 percent by weight based on a total weight of a lubricant composition containing the additive.

### TECHNICAL FIELD

The disclosure relates to lubricant compositions and in particular to additives for improving reducing the amount of intake valve deposits that form adjacent intake valves of a spark ignition direct injection (SIDI) engine.

### BACKGROUND AND SUMMARY

Spark ignition direct injection (SIDI) engines have been investigated for benefits in fuel economy and reduction in CO₂ emissions for over 90 years. Technical challenges have included fuel management control, exhaust emissions control, injector fouling and engine deposits. Asian and European manufacturers have all indicated a commitment to pursuing SIDI engine technology. However, SIDI engines do not have port fuel injectors to wash the deposits off the intake valves. Accordingly, there is no effective removal process for intake valves of SIDI engines and thus deposits tend to build over time. Intake valve deposits may eventually build up to a point where the valves remain open, either causing a loss of engine compression or causing catastrophic failure in the event a piston crown strikes the open valve.

Deposits may build up on the intake valves of the SIDI engines such that by about 35,000 miles the vehicle must be taken out of service and the valves cleaned through mechanical process. Until now, it was believed that the deposits in the engine arose primarily from the fuel and thus a variety of fuel additives were used in an attempt to reduce the formation of engine deposits. However, it has now been discovered, quite surprisingly, that the intake valve deposits in a SIDI engine arise primarily from the lubricant used in the engine. It is believed that oil vapors from the lubricant composition enter the intake valve ports via the positive crankcase ventilation (PCV) circuit and the vapors condense on the valves forming deposits. Accordingly, there is a need for a lubricant composition and method for reducing the amount of deposits formed on the intake valves of the SIDI engine.

With regard to the foregoing, embodiments of the disclosure provide a lubricant additive, an crankcase lubricant composition and a method for reducing intake valve deposits in a spark ignition direct injection (SIDI) engine. The lubricant additive includes an aromatic compound having a boiling point under standard atmospheric conditions of from about 190° to about 270° C. The aromatic compound is effective to reduce intake valve deposits in a SIDI engine when used in an amount ranging from about 0.1 to about 5.0 percent by weight based on a total weight of a lubricant composition containing the additive.

Use of an aromatic additive having a boiling point ranging from about 190° to 270° C. in a lubricant composition for an engine goes against the conventional wisdom that tends to avoid the use of volatile organic compounds in such lubricant compositions. Furthermore, it was not expected that a lubricant additive as described herein would be more effective than a fuel additive at reducing intake valve deposits in a SIDI engine.

An unexpected advantage of the use of the aromatic additive of the disclosed embodiments is that a SIDI engine containing the aromatic additive may be operated for more than twice the mileage of a vehicle operated without the additive without loss of engine efficiency or performance due to restricted air flow in intake valve ports of the engine. Other benefits and advantages may be evident from the following description and attached drawings.

### BRIEF DESCRIPTION OF THE DRAWINGS

Additional details and advantages of the disclosure will be set forth in part in the description which follows, and/or may be learned by practice of the disclosure in conjunction with the attached drawings, wherein:

FIG. 1 is a photograph of an intake valve and valve port from an air inlet side of the valve port for a vehicle having an SIDI engine run for 35,184 miles without an aromatic additive as described herein.

FIG. 2 is a close up photograph of the deposits on the valve stem of the intake valve of FIG. 1.

FIG. 3 is a photograph a representative intake valve and valve port for a vehicle having an SIDI engine run for 80,912 miles with an aromatic additive according to an embodiment of the disclosure.

FIG. 4 is a close up photograph of the deposits on the valve stem of the intake valve of FIG. 3.

It is to be understood that both the foregoing general description and the following detailed description are exemplary and explanatory only and are not restrictive of the disclosure, as claimed. The details and advantages of the disclosure may be realized and attained by means of the elements and combinations particularly pointed out in the appended claims.

### DETAILED DESCRIPTION OF EXEMPLARY EMBODIMENTS:

The present disclosure will now be described in the more limited aspects of embodiments thereof, including various examples of the formulation and use of the present disclosure. It will be understood that these embodiments are presented solely for the purpose of illustrating the invention and shall not be considered as a limitation upon the scope thereof.

With regard to the exemplary embodiments, the following definitions of terms are provided in order to clarify the meanings of certain terms as used herein.

As used herein, the terms “oil composition,” “lubrication composition,” “lubricating oil composition,” “lubricating oil,” “lubricant composition,” “lubricating composition,” “fully formulated lubricant composition,” and “lubricant” are considered synonymous, fully interchangeable terminology referring to the finished lubrication product comprising a major amount of a base oil plus a minor amount of an additive composition.

As used herein, the terms “additive package,” “additive concentrate,” and “additive composition” are considered synonymous, fully interchangeable terminology referring the portion of the lubricating composition excluding the major amount of base oil stock mixture.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include:
(1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical);
(2) substituted hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, amino, alkylamine, and sulfoxy);
(3) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents such as pyridyl, furyl, thienyl, and imidazolyl. In general, no more than two, for example, no more than one, non-hydrocarbon substituent will be present for every ten carbon atoms in the hydrocarbyl group; typically, there will be no non-hydrocarbon substituents in the hydrocarbyl group.

As used herein, the term “percent by weight”, unless expressly stated otherwise, means the percentage the recited component represents to the weight of the entire composition.

The terms “oil-soluble” or “dispersible” used herein may but do not necessarily indicate that the compounds or additives are soluble, dissolvable, miscible, or capable of being suspended in the oil in all proportions. The foregoing terms do mean, however, that they are, for instance, soluble or stably dispersible in oil to an extent sufficient to exert their intended effect in the environment in which the oil is employed. Moreover, the additional incorporation of other additives may also permit incorporation of higher levels of a particular additive, if desired.

Lubricating oils, engine lubricating oils, and/or crankcase lubricating oils of the present disclosure may be formulated by the addition of one or more additives, as described in detail below, to an appropriate base oil formulation. The additives may be combined with a base oil in the form of an additive package (or concentrate), may be combined individually with a base oil, or alternatively, may be added to the lubricant composition in an engine as a “booster” additive. A “booster” additive, as used herein, is an amount of additive added to a fully formulated lubricant composition that supplements or increases the amount of additive component in the lubricant composition over and above a conventional amount of the component typically present in the fully formulated lubricant composition. The fully formulated lubricant, engine lubricant, and/or crankcase lubricant may exhibit improved performance properties, based on the additives added and their respective proportions.

Engine or crankcase lubricant compositions, described herein, are used in vehicles containing spark ignition engines, particularly spark ignition direct injection engines. Such engines may be used in automotive and light duty truck applications and may be operated on fuels including, but not limited to, gasoline, alcohol-containing fuels, compressed natural gas, gas-to-liquid fuels, biofuels, flex-fuels, mixtures thereof, and the like. The disclosure may describe lubricants suitable for use as engine lubricants, such as automotive crankcase lubricants that meet or exceed the proposed ILSAC GF-5 lubricant standards. A conventional GF-5 lubricant composition may include one or more additive components selected from detergents, dispersants, friction modifiers, antioxidants, rust inhibitors, viscosity index improvers, emulsifiers, demulsifiers, corrosion inhibitors, antiwear agents, metal dihydrocarbyl dithiophosphates, ash-free amine phosphate salts, antifoam agents, and pour point depressants. According to an embodiment of the disclosure the lubricant composition also includes an aromatic compound in an amount that is effective to reduce intake valve deposits in a SIDI engine.

### Aromatic Additive

According to an embodiment of the disclosure, a relatively volatile aromatic additive is combined with a fully formulated lubricant composition having a boiling point under standard atmospheric conditions of from about 190° to about 270° C., wherein the aromatic compound is effective to reduce intake valve deposits in a SIDI engine when used in an amount ranging from about 0.1 to about 5.0 percent by weight based on a total weight of a lubricant composition containing the additive.

Aromatic additive compounds that may be used include compounds of the formula:

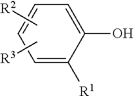

wherein each of R¹, R², and R³ is selected from hydrogen and a hydrocarbyl group containing from 1 to 6 carbon atoms, provided that at least one of R¹, R², and R³ is a hydrocarbyl group containing from 1 to 6 carbon atoms, wherein the compound has a boiling point under standard atmospheric conditions ranging from about 190° to about 270° C. Standard atmospheric conditions are room temperature and one atmosphere of pressure.

Accordingly, suitable aromatic compounds that may be used to reduce valve deposits in SIDI engines include, but are not limited to, 2,6-di-tert-butylphenol, 2,6-di-tert-butyl-4-methylphenol, 2-tert-butyl-6-methylphenol, 2-tert-butylphenol, 4-tert-butylphenol, o-cresol, m-cresol, p-cresol, and mixtures of two or more of the foregoing. Of the foregoing, particularly suitable compounds include hindered phenol compounds having a boiling point within the range of from about 190° to about 270° C., for example from about 220° to about 265° C. Examples of such compounds include 2,6-di-tert-butylphenol, 2,6-di-tert-butyl-4-methylphenol, 2-tert-butyl-6-methylphenol, 2-tert-butylphenol, and 4-tert-butylphenol.

Compared to conventional phenolic compounds used in lubricant compositions, the compounds described herein are relatively more volatile than the aromatic compounds conventionally used in lubricant composition. Without desiring to be limited by theoretical considerations, it is believed that the aromatic compound described herein may more readily volatilize and enter the intake air manifold of the SIDI engine with oil mist and vapors in the PCV circuit of the engine. As the entrained oil mist and vapor containing the aromatic condense on the intake valve stem and tulip the aromatic compound may prevent the oil from the mist and vapor from polymerizing, allowing sufficient time for the oil to naturally vaporize and be consumed in the combustion process.

The amount of aromatic additive compound in the lubricant composition is desirably an amount sufficient to maintain the performance and/or fuel economy of an SIDI engine for more than about 35,000 miles of engine operation. Accordingly, the amount of aromatic additive that may be used in a fully formulated lubricant composition for an SIDI engine may range from about 0.1 to about 5.0 percent by weight based on a total weight of a lubricant composition containing the additive. A particularly suitable amount of additive may range from about 0.5 to about 2.0 percent by weight based on a total weight of the lubricant composition containing the additive.

The aromatic additive may be initially present in a fully formulated lubricant composition, or may be added to a lubricant composition or to the crankcase of an engine containing a fully formulated lubricant composition. In another embodiment, the aromatic additive may be added to the crankcase of an engine after the engine has been operated for a predetermined number of miles in order to reduce the amount of deposits formed in the intake valves of the engine.

### Base Oil

Base oils suitable for use in formulating engine lubricant compositions may be selected from any of suitable synthetic or mineral oils or mixtures thereof. Mineral oils may include animal oils and vegetable oils (e.g., castor oil, lard oil) as well as mineral lubricating oils such as liquid petroleum oils and solvent treated or acid-treated mineral lubricating oils of the paraffinic, naphthenic or mixed paraffinic-naphthenic types. Oils derived from coal or shale may also be suitable. The base oil typically may have a viscosity of about 2 to about 15 cSt or, as a further example, about 2 to about 10 cSt at 100° C. Further, an oil derived from a gas-to-liquid process is also suitable.

Suitable synthetic base oils may include alkyl esters of dicarboxylic acids, polyglycols and alcohols, poly-alpha-olefins, including polybutenes, alkyl benzenes, organic esters of phosphoric acids, and polysilicone oils. Synthetic oils include hydrocarbon oils such as polymerized and interpolymerized olefins (e.g., polybutylenes, polypropylenes, propylene isobutylene copolymers, etc.); poly(1-hexenes), poly-(1-octenes), poly(1-decenes), etc. and mixtures thereof; alkylbenzenes (e.g., dodecylbenzenes, tetradecylbenzenes, di-nonylbenzenes, di-(2-ethylhexyl)benzenes, etc.); polyphenyls (e.g., biphenyls, terphenyl, alkylated polyphenyls, etc.); alkylated diphenyl ethers and alkylated diphenyl sulfides and the derivatives, analogs and homologs thereof and the like.

Alkylene oxide polymers and interpolymers and derivatives thereof where the terminal hydroxyl groups have been modified by esterification, etherification, etc., constitute another class of known synthetic oils that may be used. Such oils are exemplified by the oils prepared through polymerization of ethylene oxide or propylene oxide, the alkyl and aryl ethers of these polyoxyalkylene polymers (e.g., methyl-polyisopropylene glycol ether having an average molecular weight of about 1000, diphenyl ether of polyethylene glycol having a molecular weight of about 500-1000, diethyl ether of polypropylene glycol having a molecular weight of about 1000-1500, etc.) or mono- and polycarboxylic esters thereof, for example, the acetic acid esters, mixed C₃-C₈ fatty acid esters, or the C₁₃ oxo-acid diester of tetraethylene glycol.

Another class of synthetic oils that may be used includes the esters of dicarboxylic acids (e.g., phthalic acid, succinic acid, alkyl succinic acids, alkenyl succinic acids, maleic acid, azelaic acid, suberic acid, sebacic acid, fumaric acid, adipic acid, linoleic acid dimer, malonic acid, alkyl malonic acids, alkenyl malonic acids, etc.) with a variety of alcohols (e.g., butyl alcohol, hexyl alcohol, dodecyl alcohol, 2-ethylhexyl alcohol, ethylene glycol, diethylene glycol monoether, propylene glycol, etc.) Specific examples of these esters include dibutyl adipate, di(2-ethylhexyl)sebacate, di-n-hexyl fumarate, dioctyl sebacate, diisooctyl azelate, diisodecyl azelate, dioctyl phthalate, didecyl phthalate, dieicosyl sebacate, the 2-ethylhexyl diester of linoleic acid dimer, the complex ester formed by reacting one mole of sebacic acid with two moles of tetraethylene glycol and two moles of 2-ethylhexanoic acid and the like.

Esters useful as synthetic oils also include those made from C₅ to C₁₂ monocarboxylic acids and polyols and polyol ethers such as neopentyl glycol, trimethylol propane, pentaerythritol, dipentaerythritol, tripentaerythritol, etc.

Hence, the base oil used which may be used to make the engine lubricant compositions as described herein may be selected from any of the base oils in Groups I-V as specified in the American Petroleum Institute (API) Base Oil Interchangeability Guidelines. Such base oil groups are as follows:

TABLE 1

| Base Oil | Sulfur | | Saturates | Viscosity |
|---|---|---|---|---|
| Group¹ | (wt %) | | (wt %) | Index |
| Group I | >0.03 | And/or | <90 | 80 to 120 |
| Group II | ≦0.03 | And | ≧90 | 80 to 120 |
| Group III | ≦0.03 | And | ≧90 | ≧120 |

| | |
|---|---|
| Group IV | all polyalphaolefins (PAOs) |
| Group V | all others not included in Groups I-IV |

¹Groups I-III are mineral oil base stocks.

The base oil may contain a minor or major amount of a poly-alpha-olefin (PAO). Typically, the poly-alpha-olefins are derived from monomers having from about 4 to about 30, or from about 4 to about 20, or from about 6 to about 16 carbon atoms. Examples of useful PAOs include those derived from octene, decene, mixtures thereof, and the like. PAOs may have a viscosity of from about 2 to about 15, or from about 3 to about 12, or from about 4 to about 8 cSt at 100° C. Examples of PAOs include 4 cSt at 100° C. poly-alpha-olefins, 6 cSt at 100° C. poly-alpha-olefins, and mixtures thereof. Mixtures of mineral oil with the foregoing poly-alpha-olefins may be used.

The base oil may be an oil derived from Fischer-Tropsch synthesized hydrocarbons. Fischer-Tropsch synthesized hydrocarbons are made from synthesis gas containing H₂ and CO using a Fischer-Tropsch catalyst. Such hydrocarbons typically require further processing in order to be useful as the base oil. For example, the hydrocarbons may be hydroisomerized using processes disclosed in U.S. Pat. Nos. 6,103,099 or 6,180,575; hydrocracked and hydroisomerized using processes disclosed in U.S. Pat. Nos. 4,943,672 or 6,096,940; dewaxed using processes disclosed in U.S. Pat. No. 5,882,505; or hydroisomerized and dewaxed using processes disclosed in U.S. Pat. Nos. 6,013,171; 6,080,301; or 6,165,949.

Unrefined, refined, and rerefined oils, either mineral or synthetic (as well as mixtures of two or more of any of these) of the type disclosed hereinabove can be used in the base oils. Unrefined oils are those obtained directly from a mineral or synthetic source without further purification treatment. For example, a shale oil obtained directly from retorting operations, a petroleum oil obtained directly from primary distillation or ester oil obtained directly from an esterification process and used without further treatment would be an unrefined oil. Refined oils are similar to the unrefined oils except they have been further treated in one or more purification steps to improve one or more properties. Many such purification techniques are known to those skilled in the art such as solvent extraction, secondary distillation, acid or base extraction, filtration, percolation, etc. Rerefined oils are obtained by processes similar to those used to obtain refined oils applied to refined oils which have been already used in service. Such rerefined oils are also known as reclaimed or reprocessed oils and often are additionally processed by techniques directed to removal of spent additives, contaminants, and oil breakdown products.

The base oil may be combined with an additive composition as disclosed in embodiments herein to provide an engine lubricant composition suitable for the crankcase of the engine. Accordingly, the base oil may be present in the engine lubricant composition in an amount ranging from about 50 wt % to about 95 wt % based on a total weight of the lubricant composition.

### Dispersant

Dispersants contained in fully formulated lubricant compositions according to the disclosure may include, but are not limited to, an oil soluble polymeric hydrocarbon backbone having functional groups that are capable of associating with particles to be dispersed. Typically, the dispersants comprise amine, alcohol, amide, or ester polar moieties attached to the polymer backbone often via a bridging group. Dispersants may be selected from Mannich dispersants as described in U.S. Pat. Nos. 3,697,574 and 3,736,357; ashless succinimide dispersants as described in U.S. Pat. Nos. 4,234,435 and 4,636,322; amine dispersants as described in U.S. Pat. Nos. 3,219,666, 3,565,804, and 5,633,326; Koch dispersants as described in U.S. Pat. Nos. 5,936,041, 5,643,859, and 5,627,259, and polyalkylene succinimide dispersants as described in U.S. Pat. Nos. 5,851,965; 5,853,434; and 5,792,729.

A suitable dispersant that may be used in a fully formulated lubricant composition may include a reaction product of A) a hydrocarbyl-carboxylic acid or anhydride or a hydrocarbyl-substituted Mannich base and B) a polyamine containing at least two nitrogen atoms. The hydrocarbyl moiety of the hydrocarbyl-carboxylic acid or anhydride of Component A may be derived from butene polymers, for example polymers of isobutylene. Suitable polyisobutenes for use herein include those formed from polyisobutylene or highly reactive polyisobutylene having at least about 60%, such as about 70% to about 90% and above, terminal vinylidene content. Suitable polyisobutenes may include those prepared using BF₃ catalysts. The number average molecular, weight of the polyalkenyl substituent may vary over a wide range, for example from about 100 to about 5000, such as from about 500 to about 5000, as determined by GPC as described above.

The carboxylic acid or anhydride of Component A may be selected from carboxylic reactants other than maleic anhydride, such as maleic acid, fumaric acid, malic acid, tartaric acid, itaconic acid, itaconic anhydride, citraconic acid, citraconic anhydride, mesaconic acid, ethylmaleic anhydride, dimethylmaleic anhydride, ethylmaleic acid, dimethylmaleic acid, hexylmaleic acid, and the like, including the corresponding acid halides and lower aliphatic esters. A mole ratio of maleic anhydride to hydrocarbyl moiety in a reaction mixture used to make Component A may vary widely. Accordingly, the mole ratio may vary from about 5:1 to about 1.5, for example from about 3:1 to about 1:3, and as a further example, the maleic anhydride may be used in stoichiometric excess to force the reaction to completion. The unreacted maleic anhydride may be removed by vacuum distillation.

Any of numerous polyamines can be used as Component B in preparing the functionalized dispersant. Non-limiting exemplary polyamines may include aminoguanidine bicarbonate (AGBC), diethylene triamine (DETA), triethylene tetramine (TETA), tetraethylene pentamine (TEPA), pentaethylene hexamine (PEHA) and heavy polyamines. A heavy polyamine may comprise a mixture of polyalkylenepolyamines having small amounts of lower polyamine oligomers such as TEPA and PEHA, but primarily oligomers having seven or more nitrogen atoms, two or more primary amines per molecule, and more extensive branching than conventional polyamine mixtures. Additional non-limiting polyamines which may be used to prepare the hydrocarbyl-substituted succinimide dispersant are disclosed in U.S. Pat. No. 6,548,458, the disclosure of which is incorporated herein by reference in its entirety. In an embodiment of the disclosure, the polyamine may be selected from tetraethylene pentamine (TEPA).

The fully formulated lubricant composition may contain from about 0.5 weight percent to about. 10.0 weight of the dispersant described above based on a total weight of the lubricant composition. A typical range of dispersant may be from about 2 weight percent to about 5 weight percent based on a total weight of the lubricant composition.

### Metal-Containing Detergents

Metal detergents that may be used with the dispersant reaction product described above generally comprise a polar head with a long hydrophobic tail where the polar head comprises a metal salt of an acidic organic compound. The salts may contain a substantially stoichiometric amount of the metal, in which case they are usually described as normal or neutral salts, and would typically have a total base number or TBN (as measured by ASTM D2896) of from about 0 to less than about 150. Large amounts of a metal base may be included by reacting an excess of a metal compound such as an oxide or hydroxide with an acidic gas such as carbon dioxide. The resulting overbased detergent comprises micelles of neutralized detergent surrounding a core of inorganic metal base (e.g., hydrated carbonates). Such overbased detergents may have a TBN of about 150 or greater, such as from about 150 to about 450 or more.

Detergents that may be suitable for use in the present embodiments include oil-soluble overbased, low base, and neutral sulfonates, phenates, sulfurized phenates, and salicylates of a metal, particularly the alkali or alkaline earth metals, e.g., sodium, potassium, lithium, calcium, and magnesium. More than one metal may be present, for example, both calcium and magnesium. Mixtures of calcium and/or magnesium with sodium may also be suitable. Suitable metal detergents may be overbased calcium or magnesium sulfonates having a TBN of from 150 to 450 TBN, overbased calcium or magnesium phenates or sulfurized phenates having a TBN of from 150 to 300 TBN, and overbased calcium or magnesium salicylates having a TBN of from 130 to 350. Mixtures of such salts may also be used.

The metal-containing detergent may be present in a lubricating composition in an amount of from about 0.5 wt % to about 5 wt %. As a further example, the metal-containing detergent may be present in an amount of from about 1.0 wt % to about 3.0 wt %. The metal-containing detergent may be present in a lubricating composition in an amount sufficient to provide from about 500 to about 5000 ppm alkali and/or alkaline earth metal to the lubricant composition based on a total weight of the lubricant composition. As a further example, the metal-containing detergent may be present in a lubricating composition in an amount sufficient to provide from about 1000 to about 3000 ppm alkali and/or alkaline earth metal.

### Phosphorus-Based Antiwear Agents

Phosphorus-based wear preventative agents may be used and may comprise a metal dihydrocarbyl dithiophosphate compound, such as but not limited to a zinc dihydrocarbyl dithiophosphate compound. Suitable metal dihydrocarbyl dithiophosphates may comprise dihydrocarbyl dithiophosphate metal salts wherein the metal may be an alkali or alkaline earth metal, or aluminum, lead, tin, molybdenum, manganese, nickel, copper, or zinc.

Dihydrocarbyl dithiophosphate metal salts may be prepared in accordance with known techniques by first forming a dihydrocarbyl dithiophosphoric acid (DDPA), usually by reaction of one or more alcohol or a phenol with P₂S₅ and then neutralizing the formed DDPA with a metal compound. For example, a dithiophosphoric acid may be made by reacting mixtures of primary and secondary alcohols. Alternatively, multiple dithiophosphoric acids can be prepared where the hydrocarbyl groups on one are entirely secondary in character and the hydrocarbyl groups on the others are entirely primary in character. To make the metal salt, any basic or neutral metal compound could be used but the oxides, hydroxides and carbonates are most generally employed. Commercial additives frequently contain an excess of metal due to the use of an excess of the basic metal compound in the neutralization reaction.

The zinc dihydrocarbyl dithiophosphates (ZDDP) are oil soluble salts of dihydrocarbyl dithiophosphoric acids and may be represented by the following formula:

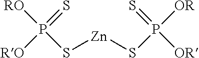

wherein R and R′ may be the same or different hydrocarbyl radicals containing from 1 to 18, for example 2 to 12, carbon atoms and including radicals such as alkyl, alkenyl, aryl, arylalkyl, alkaryl, and cycloaliphatic radicals. R and R′ groups may be alkyl groups of 2 to 8 carbon atoms. Thus, the radicals may, for example, be ethyl, n-propyl, i-propyl, n-butyl, i-butyl, sec-butyl, amyl, n-hexyl, i-hexyl, n-octyl, decyl, dodecyl, octadecyl, 2-ethylhexyl, phenyl, butylphenyl, cyclohexyl, methylcyclopentyl, propenyl, butenyl. In order to obtain oil solubility, the total number of carbon atoms (i.e., R and R′) in the dithiophosphoric acid will generally be about 5 or greater. The zinc dihydrocarbyl dithiophosphate can therefore comprise zinc dialkyl dithiophosphates.

Other suitable components that may be utilized as the phosphorus-based wear preventative include any suitable organophosphorus compound, such as but not limited to, phosphates, thiophosphates, di-thiophosphates, phosphites, and salts thereof and phosphonates. Suitable examples are tricresyl phosphate (TCP), di-alkyl phosphite (e.g., dibutyl hydrogen phosphite), and amyl acid phosphate.

Another suitable component is a phosphorylated succinimide such as a completed reaction product from a reaction between a hydrocarbyl substituted succinic acylating agent and a polyamine combined with a phosphorus source, such as inorganic or organic phosphorus acid or ester. Further, it may comprise compounds wherein the product may have amide, amidine, and/or salt linkages in addition to the imide linkage of the type that results from the reaction of a primary amino group and an anhydride moiety.

The phosphorus-based wear preventative may be present in a lubricating composition in an amount sufficient to provide from about 200 to about 2000 ppm phosphorus. As a further example, the phosphorus-based wear preventative may be present in a lubricating composition in an amount sufficient to provide from about 500 to about 800 ppm phosphorus.

The phosphorus-based wear preventative may be present in a lubricating composition in an amount sufficient to provide a ratio of alkali and/or alkaline earth metal content (ppm) based on the total amount of alkali and/or alkaline earth metal in the lubricating composition to phosphorus content (ppm) based on the total amount of phosphorus in the lubricating composition of from about 1.6 to about 3.0 (ppm/ppm).

### Friction Modifiers

Embodiments of the present disclosure may include one or more friction modifiers. Suitable friction modifiers may comprise metal containing and metal-free friction modifiers and may include, but are not limited to, imidazolines, amides, amines, succinimides, alkoxylated amines, alkoxylated ether amines, amine oxides, amidoamines, nitriles, betaines, quaternary amines, imines, amine salts, amino guanadine, alkanolamides, phosphonates, metal-containing compounds, glycerol esters, and the like.

Suitable friction modifiers may contain hydrocarbyl groups that are selected from straight chain, branched chain, or aromatic hydrocarbyl groups or admixtures thereof, and may be saturated or unsaturated. The hydrocarbyl groups may be composed of carbon and hydrogen or hetero atoms such as sulfur or oxygen. The hydrocarbyl groups may range from about 12 to about 25 carbon atoms and may be saturated or unsaturated.

Aminic friction modifiers may include amides of polyamines. Such compounds can have hydrocarbyl groups that are linear, either saturated or unsaturated, or a mixture thereof and may contain from about 12 to about 25 carbon atoms.

Further examples of suitable friction modifiers include alkoxylated amines and alkoxylated ether amines. Such compounds may have hydrocarbyl groups that are linear, either saturated, unsaturated, or a mixture thereof. They may contain from about 12 to about 25 carbon atoms. Examples include ethoxylated amines and ethoxylated ether amines.

The amines and amides may be used as such or in the form of an adduct or reaction product with a boron compound such as a boric oxide, boron halide, metaborate, boric acid or a mono-, di- or tri-alkyl borate. Other suitable friction modifiers are described in U.S. Pat. No. 6,300,291, herein incorporated by reference.

Other suitable friction modifiers may include an organic, ashless (metal-free), nitrogen-free organic friction modifier. Such friction modifiers may include esters formed by reacting carboxylic acids and anhydrides with alkanols. Other useful friction modifiers generally include a polar terminal group (e.g. carboxyl or hydroxyl) covalently bonded to an oleophilic hydrocarbon chain. Esters of carboxylic acids and anhydrides with alkanols are described in U.S. Pat. No. 4,702,850. Another example of an organic ashless nitrogen-free friction modifier is known generally as glycerol monooleate (GMO) which may contain mono- and diesters of oleic acid. Other suitable friction modifiers are described in U.S. Pat. No. 6,723,685, herein incorporated by reference. The ashless friction modifier may be present in the lubricant composition in an amount ranging from about 0.1 to about 0.4 percent by weight based on a total weight of the lubricant composition.

Suitable friction modifiers may also include one or more molybdenum compounds. The molybdenum compound may be selected from the group consisting of molybdenum dithiocarbamates (MoDTC), molybdenum dithiophosphates, molybdenum dithiophosphinates, molybdenum xanthates, molybdenum thioxanthates, molybdenum sulfides, a trinuclear organo-molybdenum compound, molybdenum/amine complexes, and mixtures thereof.

Additionally, the molybdenum compound may be an acidic molybdenum compound. Included are molybdic acid, ammonium molybdate, sodium molybdate, potassium molybdate, and other alkaline metal molybdates and other molybdenum salts, e.g., hydrogen sodium molybdate, MoOCl₄, MoO₂Br₂, Mo₂O₃Cl₆, molybdenum trioxide or similar acidic molybdenum compounds. Alternatively, the compositions can be provided with molybdenum by molybdenum/sulfur complexes of basic nitrogen compounds as described, for example, in U.S. Pat. Nos. 4,263,152; 4,285,822; 4,283,295; 4,272,387; 4,265,773; 4,261,843; 4,259,195 and 4,259,194; and WO 94/06897.

Suitable molybdenum dithiocarbamates may be represented by the formula:

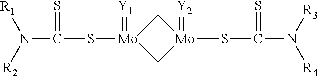

where R₁, R₂, R₃, and R₄ each independently represent a hydrogen atom, a C₁ to C₂₀ alkyl group, a C₆ to C₂₀ cycloalkyl, aryl, alkylaryl, or aralkyl group, or a C₃ to C₂₀ hydrocarbyl group containing an ester, ether, alcohol, or carboxyl group; and X₁, X₂, Y₁, and Y₂ each independently represent a sulfur or oxygen atom.

Examples of suitable groups for each of R₁, R₂, R₃, and R₄ include 2-ethylhexyl, nonylphenyl, methyl, ethyl, n-propyl, iso-propyl, n-butyl, t-butyl, n-hexyl, n-octyl, nonyl, decyl, dodecyl, tridecyl, lauryl, oleyl, linoleyl, cyclohexyl and phenylmethyl. R₁ to R₄ may each have C₆ to C₁₈ alkyl groups. X₁ and X₂ may be the same, and Y₁ and Y₂ may be the same. X₁ and X₂ may both comprise sulfur atoms, and Y₁ and Y₂ may both comprise oxygen atoms.

Further examples of molybdenum dithiocarbamates include C₆-C₁₈ dialkyl or diaryldithiocarbamates, or alkyl-aryldithiocarbamates such as dibutyl-, diamyl-di-(2-ethyl-hexyl)-, dilauryl-, dioleyl-, and dicyclohexyl-d ithiocarbamate.

Another class of suitable organo-molybdenum compounds are trinuclear molybdenum compounds, such as those of the formula Mo₃SₖLₙQ_{z} and mixtures thereof, wherein L represents independently selected ligands having organo groups with a sufficient number of carbon atoms to render the compound soluble or dispersible in the oil, n is from 1 to 4, k varies from 4 through 7, Q is selected from the group of neutral electron donating compounds such as water, amines, alcohols, phosphines, and ethers, and z ranges from 0 to 5 and includes non-stoichiometric values. At least 21 total carbon atoms may be present among all the ligands' organo groups, such as at least 25, at least 30, or at least 35 carbon atoms. Additional suitable molybdenum compounds are described in U.S. Pat. No. 6,723,685, herein incorporated by reference.

The molybdenum compound may be present in a fully formulated engine lubricant in an amount to provide about 5 ppm to 800 ppm molybdenum. As a further example, the molybdenum compound may be present in an amount to provide about 30 to 100 ppm molybdenum.

Titanium compounds may also be included in the lubricant compositions as friction modifiers. The titanium compounds include the reaction product of titanium alkoxide, such as titanium isopropoxide, and a carboxylic acid containing from 6 to 25 carbon atoms, as generally described in U.S. Pat. Nos. 7,615,519; 7,615,520; 7,709,423; 7,776,800; 7,767,632; 7,772,167; 7,879,774; 7,897,548; 8,008,237; 8,048,834, the disclosures of which are incorporated herein by reference thereto.

### Anti-Foam Agents

In some embodiments, a foam inhibitor may form another component suitable for use in the compositions. Foam inhibitors may be selected from silicones, polyacrylates, and the like. The amount of antifoam agent in the engine lubricant formulations described herein may range from about 0.001 wt % to about 0.1 wt % based on the total weight of the formulation. As a further example, antifoam agent may be present in an amount from about 0.004 wt % to about 0.008 wt %.

### Oxidation Inhibitor Components

Oxidation inhibitors or antioxidants reduce the tendency of base stocks to deteriorate in service which deterioration can be evidenced by the products of oxidation such as sludge and varnish-like deposits that deposit on metal surfaces and by viscosity growth of the finished lubricant. Such oxidation inhibitors include hindered phenols, sulfurized hindered phenols, alkaline earth metal salts of alkylphenolthioesters having C₅ to C₁₂ alkyl side chains, sulfurized alkylphenols, metal salts of either sulfurized or nonsulfurized alkylphenols, for example calcium nonylphenol sulfide, ashless oil soluble phenates and sulfurized phenates, phosphosulfurized or sulfurized hydrocarbons, phosphorus esters, metal thiocarbamates, and oil soluble copper compounds as described in U.S. Pat. No. 4,867,890.

Additional antioxidants that may be used include sterically hindered phenols and esters thereof, diarylamines, alkylated phenothiazines, sulfurized compounds, and ashless dialkyldithiocarbamates. Non-limiting examples of sterically hindered phenols include, but are not limited to, 4-ethyl-2,6-di-tertiary butylphenol, 4-propyl-2,6-di-tertiary butylphenol, 4-butyl-2,6-di-tertiary butylphenol, 4-pentyl-2,6-di-tertiary butylphenol, 4-hexyl-2,6-di-tertiary butylphenol, 4-heptyl-2,6-di-tertiary butylphenol, 4-(2-ethylhexyl)-2,6-di-tertiary butylphenol, 4-octyl-2,6-di-tertiary butylphenol, 4-nonyl-2,6-di-tertiary butylphenol, 4-decyl-2,6-di-tertiary butylphenol, 4-undecyl-2,6-di-tertiary butylphenol, 4-dodecyl-2,6-di-tertiary butylphenol, methylene bridged sterically hindered phenols including but not limited to 4,4-methylenebis(6-tert-butyl-o-cresol), 4,4-methylenebis(2-tert-amyl-o-cresol), 2,2-methylenebis(4-methyl-6 tert-butylphenol, 4,4-methylene-bis(2,6-di-tert-butylphenol) and mixtures thereof as described in U.S Publication No. 2004/0266630. The hindered phenol compounds described above may be used in addition to the aromatic compounds described herein for the purposes of improving the antioxidant properties of the lubricant without affecting intake valve deposits. In other words, the foregoing antioxidants are used in an amount that provides antioxidant effects without reducing the amount of deposits on intake valves of an SIDI engine.

Diarylamine antioxidants include, but are not limited to diarylamines having the formula:

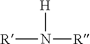

wherein R′ and R″ each independently represents a substituted or unsubstituted aryl group having from 6 to 30 carbon atoms. Illustrative of substituents for the aryl group include aliphatic hydrocarbon groups such as alkyl having from 1 to 30 carbon atoms, hydroxy groups, halogen radicals, carboxylic acid or ester groups, or nitro groups.

The aryl group is preferably substituted or unsubstituted phenyl or naphthyl, particularly wherein one or both of the aryl groups are substituted with at least one alkyl having from 4 to 30 carbon atoms, preferably from 4 to 18 carbon atoms, most preferably from 4 to 9 carbon atoms. It is preferred that one or both aryl groups be substituted, e.g. mono-alkylated diphenylamine, di-alkylated diphenylamine, or mixtures of mono- and di-alkylated diphenylamines.

The diarylamines may be of a structure containing more than one nitrogen atom in the molecule. Thus the diarylamine may contain at least two nitrogen atoms wherein at least one nitrogen atom has two aryl groups attached thereto, e.g. as in the case of various diamines having a secondary nitrogen atom as well as two aryls on one of the nitrogen atoms.

Examples of diarylamines that may be used include, but are not limited to: diphenylamine; various alkylated diphenylamines; 3-hydroxydiphenylamine; N-phenyl-1,2-phenylenediamine; N-phenyl-1,4-phenylenediamine; monobutyldiphenyl-amine; dibutyldiphenylamine; monooctyldiphenylamine; dioctyldiphenylamine; monononyldiphenylamine; dinonyldiphenylamine; monotetradecyldiphenylamine; ditetradecyldiphenylamine, phenyl-alpha-naphthylamine; monooctyl phenyl-alpha-naphthylamine; phenyl-beta-naphthylamine; monoheptyldiphenylamine; diheptyl-diphenylamine; p-oriented styrenated diphenylamine; mixed butyloctyldi-phenylamine; and mixed octylstyryldiphenylamine.

The sulfur containing antioxidants include, but are not limited to, sulfurized olefins that are characterized by the type of olefin used in their production and the final sulfur content of the antioxidant. High molecular weight olefins, i.e. those olefins having an average molecular weight of 168 to 351 g/mole, are preferred. Examples of olefins that may be used include alpha-olefins, isomerized alpha-olefins, branched olefins, cyclic olefins, and combinations of these.

Alpha-olefins include, but are not limited to, any C₄ to C₂₅ alpha-olefins. Alpha-olefins may be isomerized before the sulfurization reaction or during the sulfurization reaction. Structural and/or conformational isomers of the alpha olefin that contain internal double bonds and/or branching may also be used. For example, isobutylene is a branched olefin counterpart of the alpha-olefin 1-butene.

Sulfur sources that may be used in the sulfurization reaction of olefins include: elemental sulfur, sulfur monochloride, sulfur dichloride, sodium sulfide, sodium polysulfide, and mixtures of these added together or at different stages of the sulfurization process.

Unsaturated oils, because of their unsaturation, may also be sulfurized and used as an antioxidant. Examples of oils or fats that may be used include corn oil, canola oil, cottonseed oil, grapeseed oil, olive oil, palm oil, peanut oil, coconut oil, rapeseed oil, safflower seed oil, sesame seed oil, soybean oil, sunflower seed oil, tallow, and combinations of these.

The amount of sulfurized olefin or sulfurized fatty oil delivered to the finished lubricant is based on the sulfur content of the sulfurized olefin or fatty oil and the desired level of sulfur to be delivered to the finished lubricant. For example, a sulfurized fatty oil or olefin containing 20 weight % sulfur, when added to the finished lubricant at a 1.0 weight % treat level, will deliver 2000 ppm of sulfur to the finished lubricant. A sulfurized fatty oil or olefin containing 10 weight % sulfur, when added to the finished lubricant at a 1.0 weight % treat level, will deliver 1000 ppm sulfur to the finished lubricant. It is desirable that the sulfurized olefin or sulfurized fatty oil to deliver between 200 ppm and 2000 ppm sulfur to the finished lubricant.

In general terms, a suitable engine lubricant may include additive components in the ranges listed in the following table.

TABLE 3

| | Wt. % | Wt. % |
|---|---|---|
| Component | (Broad) | (Typical) |
| Dispersant | 0.5-15.0 | 1.0-10.0 |
| Additional Dispersants | 0.0-10% | 0.0-5.0% |
| Antioxidants | 0-5.0 | 0.01-3.0 |
| Metal Detergents | 0.1-15.0 | 0.2-8.0 |
| Corrosion Inhibitor | 0-5.0 | 0-2.0 |
| Metal dihydrocarbyl | 0.1-6.0 | 0.5-4.0 |
| dithiophosphate | | |
| Antifoaming agents | 0-5.0 | 0.001-0.15 |
| Antiwear agents | 0-1.0 | 0-0.8 |
| Pour point depressant | 0.01-5.0 | 0.01-1.5 |
| Viscosity modifier | 0.01-20.00 | 0.25-10.0 |
| Friction modifiers | 0-2.0 | 0.1-1.0 |
| Base oil | Balance | Balance |
| Total | 100 | 100 |

Additional optional additives that may be included in lubricant compositions described herein include, but are not limited to, rust inhibitors, emulsifiers, demulsifiers, and oil-soluble titanium-containing additives.

Additives used in formulating the compositions described herein may be blended into the base oil individually or in various sub-combinations. However, it may be suitable to blend all of the components concurrently using an additive concentrate (i.e., additives plus a diluent, such as a hydrocarbon solvent). The use of an additive concentrate may take advantage of the mutual compatibility afforded by the combination of ingredients when in the form of an additive concentrate. Also, the use of a concentrate may reduce blending time and may lessen the possibility of blending errors.

The present disclosure provides novel lubricating oil blends specifically formulated for use as automotive engine lubricants in SIDI engines. In order to demonstrate the benefits and advantages of lubricant compositions according to the disclosure, the following non-limiting examples are provided.

### EXAMPLES

Two identically equipped 2008 Pontiac Solstice test vehicles using SIDI fuel management were employed in this evaluation. Both vehicles had previously used a conventional fully formulated engine oil in their crankcase, which is an SAE 5W-30 motor oil meeting the ILSAC GF-4 specification. Both vehicles had previously developed pronounced intake valve deposits which would lead to a loss of engine efficiency and have to be mechanically removed in a maintenance process before damaging the engine. The deposits, if left to accumulate, would have lead to eventual seizure of the intake valves in the valve guides and cause damage to the engine.

Before the start of testing the valves and ports of both vehicles were disassembled, cleaned and reassembled. In Test Vehicle 1, a baseline fully formulated engine oil, as recommended by the manufacturer of the vehicle was used. In Test Vehicle 2, an SAE 5W-30 test oil formulated to meet the ILSAC GF-4 and GF-5 requirements, and containing the aromatic compound described herein was used.

Both vehicles were operated on a Mileage Accumulation Dynamometer (MAD) according to an in-house “Quad 4” driving cycle used for testing fuel effects on combustion chamber deposits. Since the fuel did not interact with the intake valve this was not deemed to be a test variable. The evaluation was concluded at 35,184 miles when Test Vehicle 1 developed sufficient deposits to cause loss of engine efficiency due to restricted air flow in the intake port and around the intake valve. Test Vehicle 2 was still operating normally at 80912 miles.

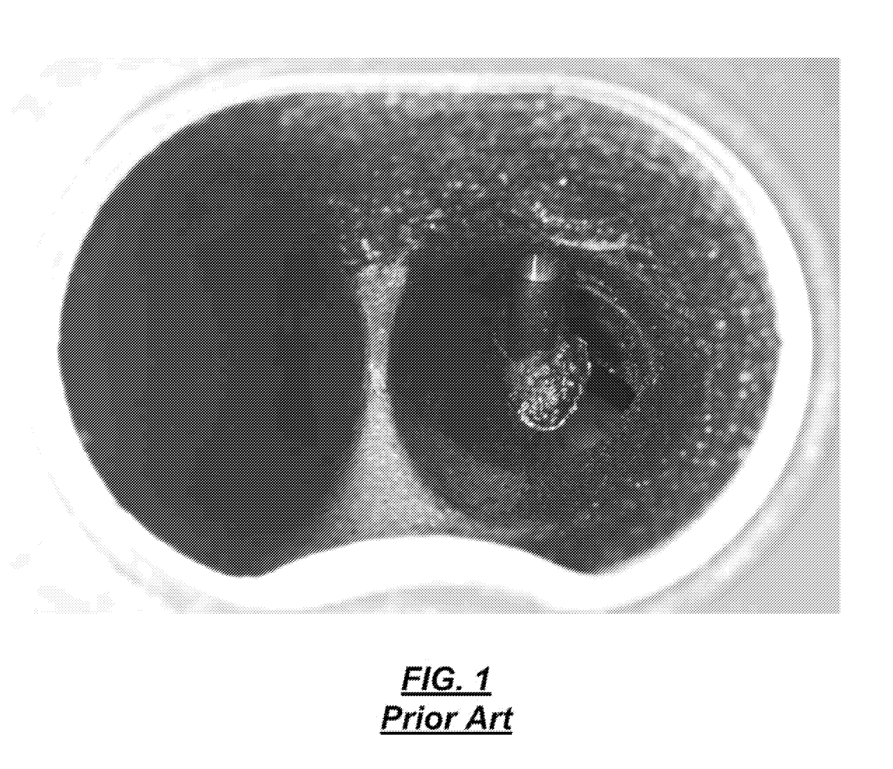

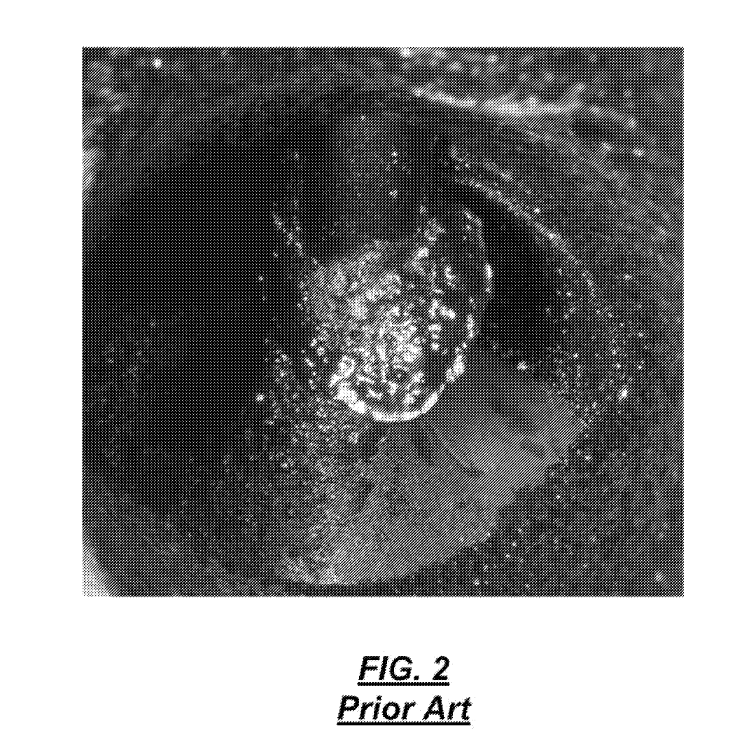

FIG. 1 is a photograph of one of the representative intake valve ports and valve stems from Test Vehicle 1 after 35,184 miles operating on the conventional engine oil composition. Because of previous experience with stuck valves the operators of this test knew that there was sufficient accumulation to indicate imminent valve sticking. FIG. 2 is a close up photograph of the deposits on the valve stem FIG. 1.

From previous experience it is known that the oily deposits shown in FIGS. 1 and 2 were derived from recirculated crankcase gas containing vaporized engine oil. Because SIDI engines do not use port fuel injection there is no fuel to wash the oily deposits from the valve stems and ports as would occur in a port fuel injected engine.

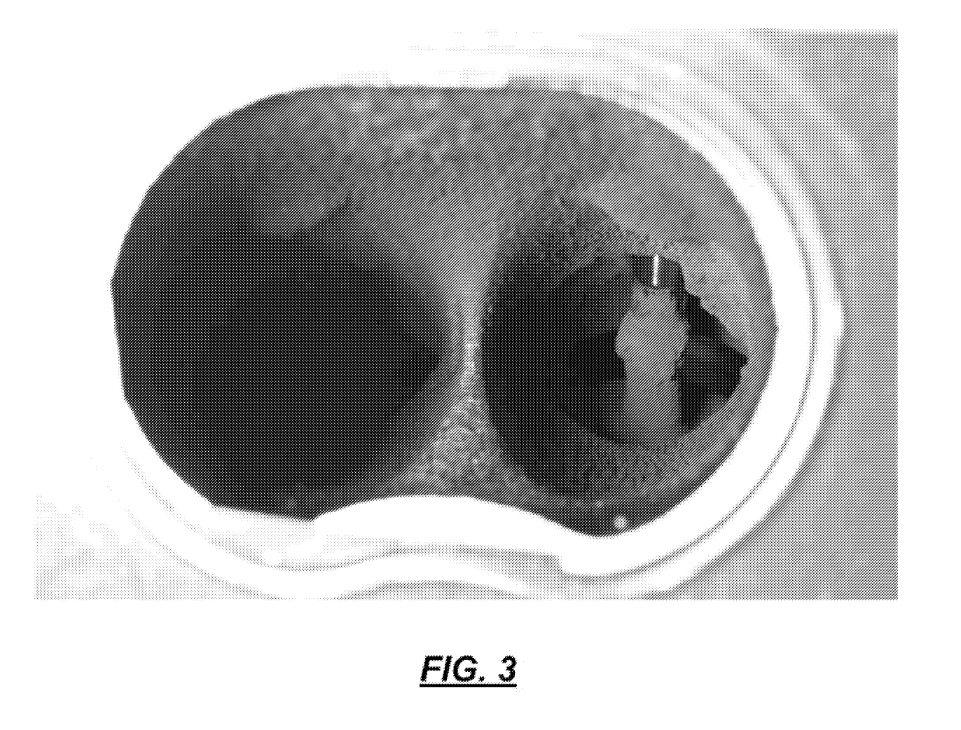

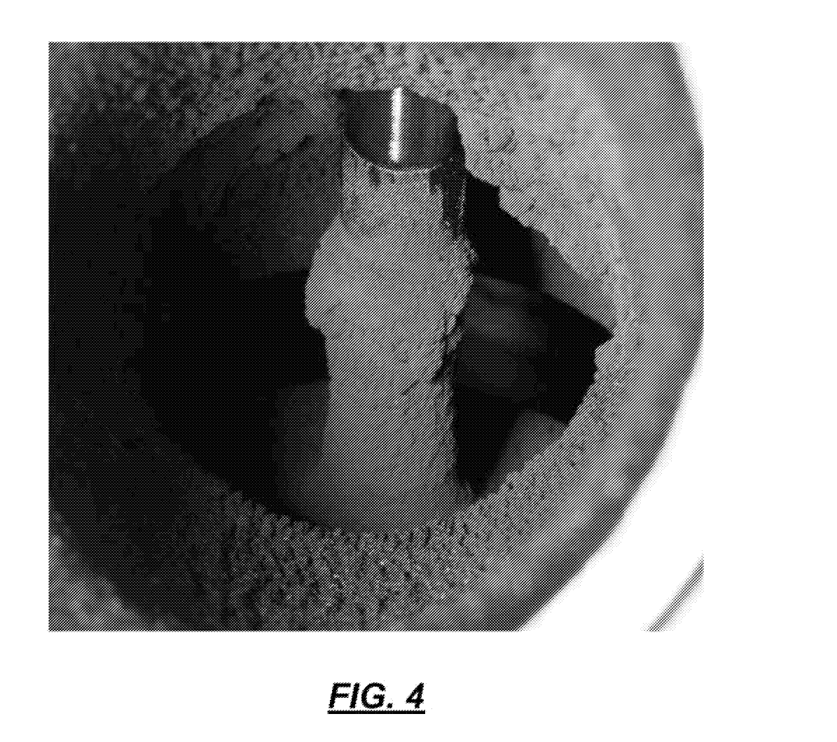

FIG. 3 is a photograph of one of the representative intake valve ports and valve stems from Vehicle 2 after running the vehicle for 80912 miles. FIG. 4 is a close-up photograph of the valve stem of FIG. 3.

As shown in FIGS. 3 and 4, the appearance of the ports and valves from the vehicle using the aromatic compound described herein were significantly different. The deposits illustrated by FIGS. 3 and 4 showed no evidence of oily deposits. By contrast to FIGS. 1 and 2, the deposits shown in FIGS. 3 and 4 have an ash-like appearance. It is believed that the ash was derived from the metallic compounds used in the oil compositions, e.g., the zinc dithio-phosphate anti-wear additive, and the calcium sulfonate detergent. As the oil portion was volatilized the ash elements were left behind. The ash-like deposit was brittle in nature and was prone to flaking off of the valve stem and valve as the valve moved up and down in the guide and port of the engine. It is believed that the aromatic compound additive described herein stabilized the oily deposits long enough to allow time for natural volatilization of the oil component in the deposits, leaving only an ash residue on the valve stem and port. By contrast, the oily deposits shown in FIGS. 1 and 2 formed larger and harder deposits that experience has taught leads to valve seizure.

At numerous places throughout this specification, reference has been made to a number of U.S. Patents. All such cited documents are expressly incorporated in full into this disclosure as if fully set forth herein.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the invention being indicated by the following claims.

The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

What is claimed is:

1. A lubricant additive effective for reducing intake valve deposits in a spark ignition direct injection (SIDI) engine comprising an aromatic compound having a boiling point under standard atmospheric conditions of from about 190° to about 270° C., wherein the aromatic compound is effective to reduce intake valve deposits in a SIDI engine when used in an amount ranging from about 0.1 to about 5.0 percent by weight based on a total weight of a lubricant composition containing the additive.

2. The lubricant additive of claim 1, wherein the aromatic compound is selected from the group consisting of 2,6-di-tert-butylphenol, 2,6-di-tert-butyl-4-methylphenol, 2-tert-butyl-6-methylphenol, 2-tert-butylphenol, 4-tert-butylphenol, o-cresol, m-cresol, p-cresol, and mixtures of two or more of the foregoing.

3. The lubricant additive of claim 1, wherein the aromatic compound comprises 2,6-di-tert-butylphenol.

4. A lubricant composition comprising from about 0.5 to about 2.0 percent by weight of the lubricant additive of claim 1.

5. A lubricant booster additive for addition to a crankcase of a SIDI engine comprising the lubricant additive of claim 1.

6. A method for reducing intake valve deposits in a spark ignition direct injection (SIDI) engine comprising providing to a crankcase of the engine a lubricant composition comprising an amount of aromatic compound having a boiling point under standard atmospheric conditions of from about 190° to about 270° C., wherein the amount of aromatic compound is sufficient to reduce intake valve deposits, and operating the engine for a period of time sufficient to vaporize at least a portion of the aromatic compound so that the aromatic compound contacts intake valves of the engine.

7. The method of claim 1, wherein the aromatic compound is selected from the group consisting of 2,6-di-tert-butylphenol, 2,6-di-tert-butyl-4-methylphenol, 2-tert-butyl-6-methylphenol, 2-tert-butylphenol, 4-tert-butylphenol, o-cresol, m-cresol, p-cresol, and mixtures of two or more of the foregoing.

8. The method of claim 7, wherein the aromatic compound comprises 2,6-di-tert-butylphenol.

9. The method of claim 6, wherein the amount of aromatic compound in the lubricant composition ranges from about 0.1 to about 5.0 percent by weight based on a total weight of a lubricant composition.

10. The method of claim 6, wherein the amount of aromatic compound in the lubricant composition ranges from about 0.5 to about 2.0 percent by weight based on a total weight of a lubricant composition.

11. A crankcase lubricant composition for a spark ignition direct injection (SIDI) engine comprising an amount of aromatic compound having a boiling point under standard atmospheric conditions of from about 190° to about 270° C., wherein the amount of aromatic compound is sufficient to reduce intake valve deposits in the SIDI engine.

12. The crankcase lubricant composition of claim 11, wherein the aromatic compound is selected from the group consisting of 2,6-di-tert-butylphenol, 2,6-di-tert-butyl-4-methylphenol, 2-tert-butyl-6-methylphenol, 2-tert-butylphenol, 4-tert-butylphenol, o-cresol, m-cresol, p-cresol, and mixtures of two or more of the foregoing.

13. The crankcase lubricant composition of claim 11, wherein the amount of aromatic compound in the lubricant composition ranges from about 0.1 to about 5.0 percent by weight based on a total weight of a lubricant composition.

14. The crankcase lubricant composition of claim 11, wherein the amount of aromatic compound in the lubricant composition ranges from about 0.5 to about 2.0 percent by weight based on a total weight of a lubricant composition.

15. The crankcase lubricant composition of claim 11, further comprising one or more of the members of the group selected from detergents, dispersants, friction modifiers, antioxidants, rust inhibitors, viscosity index improvers, emulsifiers, demulsifiers, corrosion inhibitors, antiwear agents, metal dihydrocarbyl dithiophosphates, ash-free amine phosphate salts, antifoam agents, and pour point depressants.

16. The crankcase lubricant composition of claim 11, further comprising an oil-soluble titanium-containing additive.
